(19) United States
(12) Patent Application Publication
(10) Pub. No.: US 2016/0213727 A1
(43) Pub. Date: Jul. 28, 2016

(54) Methods and Uses of High Thymoquinone and Thymohydroquinone Essential Oil of Monarda

(71) Applicant: William G. Rohlfsen, Mason City, IA (US)

(72) Inventor: William G. Rohlfsen, Mason City, IA (US)

(21) Appl. No.: 15/088,757

(22) Filed: Apr. 1, 2016

Related U.S. Application Data
(63) Continuation-in-part of application No. 15/006,611, filed on Jan. 26, 2016.
Application No. 15/006,611 is a continuation-in-part of application No. 13/804,026, filed on Mar. 14, 2013, now Pat. No. 9,073,824.
Application No. 13/804,026 is a continuation-in-part of application No. 13/359,045, filed on Jan. 26, 2012, now Pat. No. 9,029,610.

(51) Int. Cl. A61K 36/53; A61K 31/05; A61K 9/00; A61K 31/122

(52) U.S. Cl. CPC A61K 36/53; A61K 31/122; A61K 31/05; A61K 9/0073; A61K 9/0014; A61K 9/0053; A61K 2236/331

(57) ABSTRACT

A method for cultivating Monarda for production of thymoquinone and thymohydroquinone includes seeding or plug planting Monarda fistulosa and/or Monarda didyma plants. The method of cultivation results in increased production of essential oils including thymoquinone and thymohydroquinone which are believed to have anti-inflammatory effects relative to a number of health-related conditions. The invention describes ways to deliver the thymoquinone and/or thymohydroquinone rich oil to a human or animal, using a variety of therapeutic delivery methods, for the prevention or reduction of inflammation associated with a disease state.

### CROSS-REFERENCE TO RELATED APPLICATIONS

This application is a continuation in part and claims priority to U.S. patent application Ser. No. 15/006,611filed Jan. 26, 2016, which is a continuation in part and claims priority to Ser. No. 13/804,026 filed Mar. 14, 2013 which is a continuation in part of application Ser. No. 13/359,045 filed on Jan. 26, 2012 filed with the United States Patent and Trademark Office the entire contents of which are herein incorporated by reference.

### FIELD OF INVENTION

The present invention relates to methods of using oil extracted from Monarda Fistulosa The methods include treatment and/or prevention of inflammatory ailments and the symptoms associated therewith, including but not limited to cancers such as leukemia, arteriosclerosis, rheumatoid arthritis, multiple-sclerosis, and other degenerative diseases, and for use as an anti-microbial and as an antiviral.

### TECHNICAL FIELD

The present invention relates to pharmaceutical, nutraceutical and/or phytoceutical compositions which include oil extract from plants from the Monarda family, more specifically, from Monarda fistulosa.

### BACKGROUND

### Oils And Uses

Some plant essential oils have long been known to possess properties that can be employed as food or flavoring, and for medicinal and industrial purposes. Different plant species provide different oils. Examples include peppermint from the peppermint plant's leaves which provide flavoring and is sometimes used as an aid for digestive issues; clove oil extracted from flowers and used for flavoring, scent, and various historical medicinal purposes; and eucalyptus oil, produced from the leaves of Eucalyptus globulus and used in many cleaning solutions and as an antiseptic.

Although it would seem likely that every plant of a species would have a similar oil content profile, it has been found instead that different races of a plant species may provide different ratios of the essential oils they produce. There are many theories as to the reasons for these differences which include response to climate, moisture levels, nutrient levels, evolution due to these pressures, etc. Additionally, individual plants within a race may exhibit different profiles of essential oils and their constituents.

Many plants are indigenous to a particular geographic area and/or climate. Historically, those who sought plant oils would gather many plants from their natural habitat. Some plants containing desirable oil have been cultivated for research purposes related to the oil with varying levels of success.

### Monarda Fistulosa

Mint family plants have been cited in medicine journals. In addition to the folk medicinal purposes, pharmaceutical compositions have been derived from or made to include mint family essential oils or their constituents. Particularly thymol(5-methyl-2[1-methylethyl]phenol) a.k.a. isopropyl-cresol and carvacrol (5-isopropyl-2-methylphenol) a.k.a. isopropyl-o-cresol, can be synthetic or obtained from oil extracted from plants such as Origanuum vulgaris, Thymus vulgaris, Mentha piperita, Thymus sepilum, Saturia hortensis, Saturea montana, Saturea subricata, Carum corticum, Thymus zugus, Ocimum gratisimum, Moranda pungata, Mosla jananoic, Monarda didyma and Monarda fistulosa.

Monarda fistulosa and many other plants belonging to the true mint family i.e. labiate, have been a source of geraniol, thymol and carvacrol essential oils. Geraniol is used in perfumes. Thymol is used in mouthwash as an antiseptic and has been shown to have antifungal activity, as well. It is known that Carvacrol may also be used as an antimicrobial, antifungal and as a flavoring.

Monarda is a genus consisting of at least about 16 species. Monarda plants have been cited as a plant family that produces oils. The plants are erect, herbaceous, annual or perennial, in the family Lamiaceae and are native to North America. The plants typically range in height from 1 to 3 feet (0.2 to 0.9 m), and have an equal spread, with slender and long-tapering (lanceolate) leaves. The leaves, when crushed, exude a spicy, highly fragrant oil. Common names include bee balm, horsemint, and bergamot, among others. When the term “Monarda” is used herein it is understood that it includes plants known by these names, as well, provided the plant meets the characteristics of the Monarda genus. Commercial use of Monarda to date includes propagation for seeds to add color to natural prairie and wildflower seed mixes, or selected Sweet Monarda may be grown to produce geraniol used in perfumes.

Monarda fistulosa is a member of the mint family and a close cousin of Monarda didyma. As such the two variants share certain characterisitics, however, their bioactivities are not the same. The body of knowledge regarding Monarda fistulosa is not as rich as that related to the didyma. Both contain carvacrol and thymol as constituents in the essential oil. Carvacrol has a boiling point of about 233 C while thymol boils at 237-238 C. Thymol has antiseptic, biocidal, and antimicrobial properties. It is a fungicide, as well. There is some evidence that thymol has anti-tumour effects.

Carvacrol is a monoterpenic phenol and is isomeric with thymol. As stated above, carvacrol is known to have antimicrobial and antifungal properties. Carvacrol is believed to be responsible for the biological activities of oregano which include antimicrobial, antitumor, antimutagenic, antigenotoxic, analgesic, antispasmodic, antiinflammatory, angiogenic, antiparasitic, antiplatelet, AChe inhibitory, antielastase, insecticidal, antihepatotoxic and hepatoprotective activities.

The Monarda fistulosa essential oil is presently used as feed additive, in honeybee breeding and in managing gastrointestinal ailments. Interestingly, in some strains of Monarda fistulosa cultivated under certain conditions, the essential oil includes thymoquinone at substantial levels.

Thymoquinone (TQ) and Thymohydroquinone (THQ) are essential oils that are also present in the Monarda fistulosa plant (see Rohlfsen, U.S. Pat. No. 9,073,824). Thymoquinone has recently been discovered to have specific effects on some mammalian cancers and is a phytochemical found in the plant Nigella sativa and several other plants. It is an antioxidant and has been shown to have therapeutic effects which have been shown to decrease damage caused by heart, liver and kidney diseases in animal studies. Thymoquinone is also an angiogenesis inhibitor and appears to have analgesic and anti-convulsant effect; tests have shown thymoquinone kills pancreatic cancer cells and may have promise related to controlling certain kinds of epileptic seizures. Further, it has powerful anti-inflammatory effects. It has been shown to reduce mouse colon tumor cell invasion and inhibits tumor growth in animal models of colon cancer. It is theorized that TQ may affect immune function, cell pH, oxidative burse, migration and cytokine release. At least one study indicates that TQ downregulated TWIST 1 transcription factor to reduce epithelial to mesenchymal transition and thus inhibits metastasis. TQ has been shown to have influence on dividing cells of certain plant systems and on expression of Bcl2-associated athanogene-like (BAG-like) genes that might be involved during the process of cell death. BAG genes are known for the regulation of diverse physiological processes in animals, including apoptosis, tumorigenesis, stress responses, and cell division. TQ has shown potential against lung disease, arthritis, and hypercholesterolemia and as a chemopreventative agent against certain cancers.

Thymoquinone's anti-oxidant properties have been shown to protect against organ damage and possibly provide anti-cancer effects where it has been chosen to be an angiogenesis inhibitor, as well as an HDAC inhibitor with an effect on p53, Bax, bcl-2 NS P21 GENSE. It reduces mouse colon tumor cell invasion and has been shown to inhibit colon tumor growth in animals. Thymoquinone has been shown to induce apoptosis in thrombocytes, and to inhibit the MEK1/2 pathway in colorectal cancer. Reports have been published describing that TQ downregulates TWIST1 transcription factor with an inhibitory effect on metastasis in breast cancer models.

### Historic Uses of Monarda

In the case of Monarda fistulosa, several studies provide information pertinent to the present invention. In the early 1970's a new chemical race of Monarda fistulosa was discovered in Manitoba Canada and studied. (MARSHALL H, H, and R. W. SCORA.1 972. A new chemical race of Monarda fistulosa (Labiatae). Can. J. Bot. 50: 1845-1849.) Monarda fistulosa is widely distributed throughout North America and a plant had been discovered having a different scent than most others. It was dubbed “sweet Monarda” due to the scent which was the only difference discernable without chemical testing. Other Sweet Monarda plants were then found, albeit far more scarce than the dominant type. The Sweet Monarda plants were generally more scarce and did not grow at many of the study's collection sites. Typically, the sweet Monarda plants were collected on light sand and stabilized dunes.

Sweet Monarda was crossed with other Monarda. Then the oils of the crosses and of the Sweet Monarda were studied using chromatography of the leaf oil. The researchers found that oil from the nonsweet Monarda was moderately viscous and was reported clear and colorless whereas oil from Sweet Monarda smelled sweet. Chromatograms showed that oil from the Sweet Monarda included far more geraniol but far less thymol and carvacrol than the non-sweet Monarda plants tested.

Another study completed in 1993 looked at Monarda as a source of certain oils, specifically, geraniol, linalool, thymol and carvacrol. This study also presented the idea that plants of a given species but grown and maintained in different geographical regions may yield different oils. (Mazza, G., F. A. Kiehn, and H. H. Marshall. 1993. Monarda: A source of geraniol, linalool, thymol and carvacrol-rich essential oils. p. 628-631. In: J. Janick and J. E. Simon (eds.), New crops. Wiley, N.Y.)

When Monarda fistulosa is crossed with M. didyma a vigorous hybrid is produced that yields geraniol, linalool, thymol, carvacrol and other terpenes. However, multiple crosses as conducted by the study resulted in a sterile plant which had to be propagated by division. The study employed a planting rate of 10,000 plants/ha (or 24,000/acre). The study acknowledged that propagation via crown divisions would be easily achieved but, because crown division is cumbersome and inefficient, the study recommended using stem cuttings instead. The cuttings were recommended to be 10-12 cm in length with their bases dipped in rooting compound and then placed in sand in a misting chamber. Roots were reportedly produced in a week using this method and recommended to be transplanted in 14-16 days. Further, weeds were reported as being easily controlled by herbicides such as trifluralin, terbaacil, solan, and paraquat. Post harvest, hydrodistillation was employed to extract the oils which were then subjected to gas chromatography and mass spectrometry. One of the hybrids showed a high level of geraniol; this hybrid was grown for a short time in southern Alberta, Canada for geraniol production but for an unknown reason, the plants did not survive the second year.

Rust, otherwise known as Puccinia menthe, was reported as the major disease of the sterile Monarda (Mazza, 1993) causing defoliation, stem damage and degeneration of plants. Recommendations for control of rust include application of the herbicide paraquat in early spring. This article also reported variations of essential oil yields between about 0.65 and 1.2 g/100 g of fresh plant material or between about 60-125 kg of oil/ha.

The inventor has investigated medicinal uses of the various constituents of essential oil of Monarda fistulosa. Carvacrol and Thymol have both antiseptic and antimicrobial activities and have been used accordingly. It also appears that one or both may be used as an herbicide of sorts, specifically, a biochemical fungicide for control of moss and liverwort. Geraniol is used most often for its scent in products such as perfumes. Thymoquinone, as mentioned earlier, is indicated as having therapeutic effect in the treatment of some mammalian cancers and other ailments, especially inflammatory ailments.

### Cultivation

In general, cultivation of plants found natively for research or seed purposes may include transfer of the plants from their native growth area to a different geography, climate or soil type or a combination of these. Natural travel of seeds from one area to another may also result in new habitats for a given plant wherein the new habitat may include differing climate, soil, or pests from those in the original habitat. It has been noted that such transfer may result not only in different physical characteristics of the plant in response to the environmental differences, but that these changes may, in turn, result in different ratios of the oils produced by the plant.

In the present invention, it was postulated by the inventors that levels of oil, and perhaps quality of the oil or relative levels of the oil's constituents, in the Monarda fistulosa plants may be at least somewhat dependent on planting and growth methods. Finding a dependable means of growing and harvesting oil from Monarda fistulosa in a manner selective for the desired oil profile would be beneficial. Developing a method that minimized use of herbicides and pesticides was also desirable in order to produce high quality oil unadulterated by chemical use.

For the most part, historically Monarda fistulosa has been cultivated as a seed for wildflower planting purposes rather than for oil production albeit some plants have been selected for geraniol production. There is, therefore, only scant information regarding cultivation of Monarda fistulosa, generally, and none related to pushing production of any of the other constituents of the oil.

Monarda fistulosa is an erect aromatic annual or perennial plant which bears pretty pinkish/lavender flowers. It is often used in wildflower seed mixes and in prairie restoration projects. Previously recommended methods for cultivation of Monarda fistulosa include planting rates of between about 0.25 lbs/acre and 2 lbs/acre, and cutting and or splitting the plants to increase them. In terms of labor and time costs, the previous methods are not ideal. Further, none of the literature purports to claim these methods increase the average levels of certain components in the oil such as thymol, carvacrol or thymoquinone and no data describing the relative amounts of these components relative to cultivation methods have been located by the Applicant.

Plants of the mint family can be cultivated in several different ways. Seeds with a high percentage of organic phenolic compound can be planted in fine loose soil, in a sub-tropical climate. They may be watered and fertilized as necessary. Some recommendations include harvesting the plants soon after the plants begin to blossom; U.S. Pat. No. 6,649,660 recommends the plants be harvested within 24 hours after blossoming, more preferably within 12 hours after blossoming; the '660 also recommends harvesting in early morning or later evening hours when the leaves are NOT exposed to the sun and recommends that the harvested leaves and blossoms NOT be exposed to direct sunlight, cautioning that such exposure may reduce the amount of active material present in the leaves. The '660 then teaches drying the foliage for 7-8 days followed by steam distillation first at 100 C describing the oil as, typically, 3-4% thymol and 60-70% carvacrol with 26-37% impurities. The second distillation at 180-200 C, performed twice, may produce oil of purity 90% or greater.

U.S. Pat. Nos. 9,029,610 and 9,073,824 to Rohlfsen describe cultivation of monarda quite differently than the '660. Rohlfsen teaches a harvesting method that recommends leaving the plants lay in the sun for several hours in order to induce an increase in the certain qualities of the harvested oil, suggests different planting rates. Oil harvested from fields of Monarda fistulosa cultivated in this manner typically has a profile that may include up to and above 70% carvacrol, and may include up to about 5% or more thymol. The oil may also include, surprisingly, between 2% and about 40% or more thymoquinone and/or thymohydroquinone.

### Compounds and Compositions

U.S. Pat. No. 6,649,660 (previously discussed herein) discloses reacting an organic phenolic compound with organic acid to form an antimicrobial compound. Organic acids including aliphatic acids, aromatic acids, and dicarboxylic acids including common acids such as citric acid, propionic acid, fumaric acid, lactic acid, acetic acid, formic acid may be used. The reacted composition is then combined with a Group I salt resulting in an antimicrobial compound. The '660 teaches this composition may be used alone or as part of a pharmaceutical composition which includes a carrier e.g. tablet, powders, granules, capsules, ointments; or liquid form.

The '660 goes on to describe that the antimicrobial compound so formed may be used as an antimicrobial compound for treatment of: e. Coli in pigs; to address topical infections caused by Streptococcus spp., Staphylococcus spp., and Furunculose by spraying a solution on the skin; to address fungal infections and Pododermatitis-Panaricium in cows and horses caused by Streptococcus spp. Staphylococccus, by spraying a solution on an infected hoof; and to treat ear or eye inflammations, bacterial intestinal infections in humans such as those caused by E. Coli, salmonella, typhimurim, strep, staph, dysenteria, campylobacter; and to treat topical infection caused by candida, trichophytom. It also discloses capsules for release of the antimicrobial compound in the intestine (rather than the stomach). The capsules may include a powder form of the antimicrobial compound, and may combine the powder with a binder which may then be encapsulated with a coating material. One such material may be ethylcellulose and soy bean oil.

U.S. Pat. No. 8,557,306 is one of a family of patents that teach various therapeutic uses for a compound that modulates inflammation and inhibits expression of COX-2. The composition includes a fraction isolated from hops and, in some embodiments, an extract of rosemary. Rosemary extract is known to include ursolic acid along with carvacrol, and thymol. This document goes on to describe uses of the compound for the treatment and prevention of cancers, autoimmune diseases, and inflammatory diseases.

US Patent Application 2014/0255370 discloses a dietary supplement composition which includes omega-3 fatty acids, tocopherols/tocotrienols, and one or more essential oils. The supplement is described as providing health benefits including improving or supporting cardiovascular health, ocular health, cerebral and cognitive function, muscle function, and athletic performance. The disclosure describes the supplement as having pronounced anti-inflammatory benefits. The '370 application postulates a synergistic effect and teaches inclusion of a fish gelatin capsule for delivery. It mentions monarda in a long list of sources of essential oils.

US Patent Application 2013/0316432 discloses a plant extract used to inhibit activity of certain cellular proteases which otherwise degrade human tissue. The amount of inhibitory activity of the extract can be increased by stressing the plant prior to extraction of the oil; according to this application, when a plant is stressed, biochemical processes are activated and new chemicals, in addition to those constitutively expressed, are synthesized as a response, particularly defense chemicals which include protease inhibitors and antibiotics.

U.S. Pat. No. 9,040,103 describes the use of certain essential oil in prevention and therapeutic treatment of keratosis. This patent describes that UV radiation induces lesions in the DNA of epidermal cells which, if not repaired correctly, become a source of mutations that constitute the first step of tumorigenesis. Cells then lose their native response to UV, and do not effectively repair the DNA, yet the damage to the DNA does not arrest proliferation. This means replication occurs with the damaged DNA, creating lesions, increasing mutations, and genetic instability. The patent then describes the acceptance of the use of essential oils by consumers and its advantages of avoiding resistance by bacteria and virus. The patent goes on to describe that such essential oils may be obtained by CO2 extraction and solvent extractions. The '103 describes that essential oil from compact oregano comprising linalool, thymol and carvacrol can induce cell death by apoptosis in a targeted manner in cancerous human keratinocytes and pre-cancerous keratinocytes mutated at p53 compared with normal keratinocytes. The treatment does not generate inflammation since apoptosis is not a pro-inflammatory process. Normal cells are hardly affected. The composition may comprise a constituent of essential oil of Origanum compatum, in particular thymol or carvacrol. Theoretically, the essential oils cause destabilization of the mitochondrial membrane and release of reactive oxygen species, at the origin of the cytotoxicity targeted against pre-cancerous cells (HaCat cells) and cancerous cells (A431 cells) mutated at p53. This patent teaches cold extraction of the oil under high pressure, steam extraction, and dry distillation. It teaches that managed temperature and low pressure are essential in conserving aromatic quality and chemical composition. The therapeutic material may be applied via “patch” to allow contact with the affected skin area. Oral application is also contemplated.

US2010/0323041 discloses methods and compositions comprising plant extracts for the treatment of cancer. Specifically, this application provides methods and compositions that target proteases MMP-9 and cathepsin B for the purpose of inhibiting neoplastic and/or endothelial cell migration, tumor growth, tumor-induced angiogenesis and/or metastasis. This application includes disclosure for use in a nutraceutical composition. The therapeutic compositions of the invention are capable of inhibiting one or more of neoplastic cell migration, endothelial cell migration, tumour growth, tumour-induced angiogenesis and metastasis. The plant may be pre-harvest treated with water or another stressor to induce or enhance the production of extracellular protease inhibitors. Solvent extraction is described; aqueous solvent may be used. The compositions may be used as or with other anti-cancer therapeutics which prevent or delay the growth and/or metastasis of cancer cells. Application may be oral, topical, rectal or parenteral, inhalation or spray.

Other patents that use plant extracts for the treatment of cancer or for inhibiting angiogenesis include: U.S. Pat. No. 6,649,650: synergistic composition of lignans from Cedruis deodar that exhibit anticancer activities for breast, cervix, and several other cancers; U.S. Pat. No. 6,632,798: describes plant extracts comprising oleouropein to inhibit angiogenesis; US App 2004/0009239: discloses extracts of Aneoctochilus family for chemo-prevention or alternative control of certain human malignant diseases; US 2003/017334:describes plant extracts comprising a chemical agent of the diterpene family for use in the treatment or prophylaxis of prostate cancer; and US 2003/0118677: discloses use of Euphorbaciae obesa extracts to induce apoptosis and growth inhibition of a cancerous cell.

What was needed was a method of producing Monarda fistulosa on a commercial scale and economically to produce an oil comprising relatively high levels of carvacrol, and/or thymoquinone and/or thymohydroquinone. A desirable method of doing so would reduce cultivation expenses and labor while providing a dependable means for a reliable harvest of the multi-use oil and its constituents.

It was therefore one objective of the present invention to determine therapeutic uses and methods for using the essential oil of Monarda fistulosa;

It was another objective of the present invention to provide a method for cultivating Monarda fistulosa to produce essential oil pushed to comprise levels of Thymoquinone, carvacrol and/or thymol appropriate and effective for addressing respiratory symptoms, to provide anti-cancer effects, for angiogenesis purposes, to reduce inflammation caused by a variety of inflammatory diseases, to reduce inflammation in arteries, to address various cancers including, but not limited to, leukemia, lung cancer, pancreatic cancer, and liver cancer in mammals.

It was another objective of the present invention to determine and devise a number of ways to deliver the essential oil of Monarda fistulosa for the purposes of reducing inflammation, for antimicrobial purposes and for anti-viral effects.

It was a primary objective of the present invention to provide a method of cultivation of Monarda fistulosa that increased consistent results of high quality and relatively high production levels of the desired oil constituents, namely, thymoquinone, carvacrol, and/or thymol for therapeutic uses.

### SUMMARY

The present invention comprises the use of the essential oil of Monarda fistulosa sourced from plants which have been cultivated to increase the levels of Thymoquinone and/or thymohydroquinone in combination with carvacrol and thymol. The levels of thymoquinone and thymohydroquinone together in the essential oil range between about 2%_and about 40%. The uses of the oil are medicinal in nature, both therapeutic and preventative, both human and other mammals including companion animals.

The present invention includes oil of Monarda fistulosa which may be produced by using a recommended planting rate and cultivation methodology which increases germination, decreases herbicide use and fuel use, increases the yield of Monarda fistulosa oil, and increases the yield of certain essential oils such as carvacrol, thymoquinone, and thymohydroquinone, alone or in various combinations of increasing yields of these oils. Oil comprising adequate levels of these constituents is then applied or dosed in a variety of methods that allow delivery of the essential oil for therapeutic and prophylactic applications.

Although known recommended planting rates for Monarda fistulosa ranged from about 0.25 to up to 2 lbs per acre (Monarda fistulosa contains 1,120,000 seeds/lb) for producing seeds for use in wildflower seed mixes and prairie grasses, it was surprisingly found that at the rates previously recommended, weed pressure causes a decrease in yield of oil, and causes an increase in levels of unwanted contaminants associated with those weeds. These contaminants may reduce oil quality by up to 40%. Removing these weeds from a field of Monarda fistulosa grown by the prior art reduces the efficiency of the overall operation.

The inventors experimented in a 12 acre field, drill seeded at differing rates and found a dramatic decrease in weeds at the higher seeding rates versus those in the lower planting rates at the first harvest.

Alternatively, planting plugs has advantages. Unlike seeds, plug plants have a 12-14 inch base the year after the plant is planted which is similar to what could be expected from a seedling plant at year 3-4 after planting. When plugs are planted at about 24,000 plugs per acre in 24 inch rows, advantages are obtained.

The method advocates certain practices related to harvesting Monarda fistulosa for best yield of the desired oil and increase in certain constituents of that oil. The method also describes means and methods for separating the oil from the plant. Finally, the method includes recommended best practices for the maintenance of the perennial and number of seasons for production prior to destruction.

### BRIEF DESCRIPTION OF THE DRAWINGS

FIG. 1 flow chart describing the cultivation process;

FIG. 2 schematic illustrating steam distillation components.

FIG. 3 chart showing levels of constituents in the oil harvested from plants grown according to the described cultivation process;

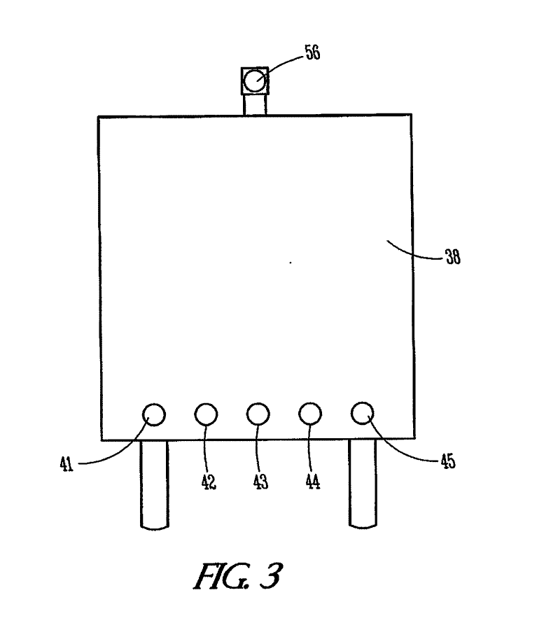

FIG. 4 chart showing effect on leukemia cells

FIG. 5 showing preliminary effect on leukemia cells, oil containing 10% thymoquinone.

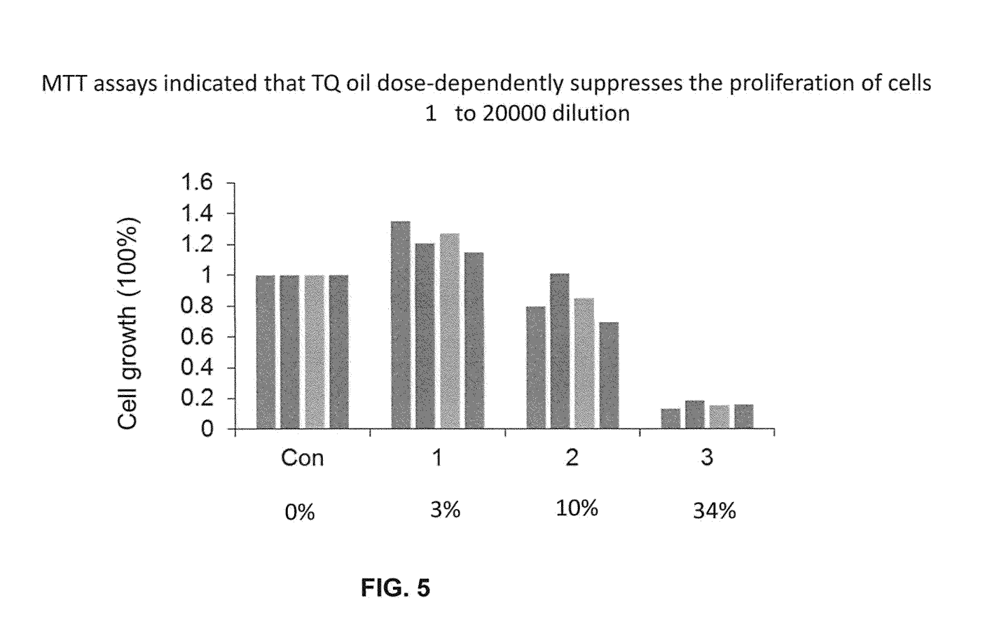

FIG. 6 showing preliminary effect on small cell lung cancer cells, oil containing 10% thymoquinone

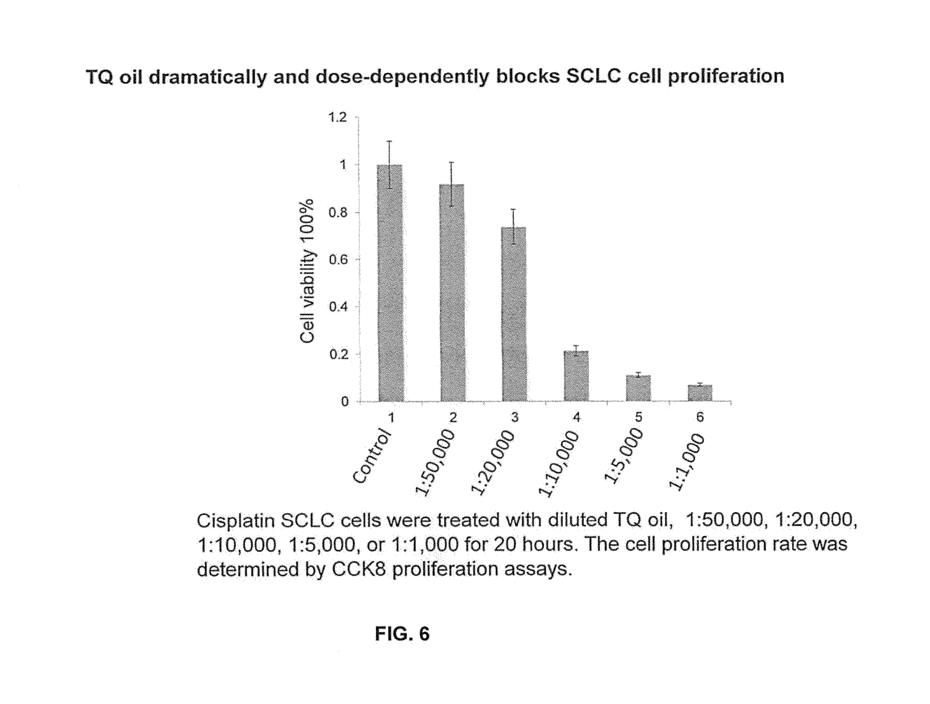

### DETAILED DESCRIPTION OF THE PREFERRED EMBODIMENTS

Specifically, this invention may be used to grow, harvest and obtain oil from at least those listed in Table 1.

TABLE 1

Monarda Didyma L.
Monarda Media Willd
Monard Menthifolia Graham
Monarda bradburiana Beck
Monarda fistulosa Sims, nom. inq.
Monarda rigida Raf.
Monarda villosa M. Martens
Monarda media Willd.
Monarda × medioides Duncan [fistulosa × media]
Monarda dressleri Scora [excluded]
Monarda fistulosa L.
Monarda fistulosa L. ssp. Fistulosa
Monarda fistulosa L. ssp. fistulosa var. fistulosa
Monarda fistulosa L. ssp. fistulosa var. menthifolia (Graham)
Fernald
Monarda fistulosa L. ssp. fistulosa var. mollis (L.) Benth.
Monarda mollis L.
Monarda scabra Beck
Monarda fistulosa L. ssp. fistulosa var. rubra A. Gray
Monarda fistulosa L. var. brevis Fosberg & Artz
Monarda fistulosa L. ssp. brevis (Fosberg & Artz) Scora, ined.

Essential oil obtained from plants grown according to the methodology disclosed herein, or other methodologies that provide oil having similar character relative to Thymoquinone, carvacrol and thymol levels, can be used for many medicinal purposes. Included in those purposes is reduction or control of inflammation which accompanies or is part of a disease state in mammals. Examples include reduction of the inflammation in arteries which is part of arteriosclerosis, and reduction of symptoms that are the result of inflammation in cancers such as, but not limited to, leukemia, liver cancer, lung cancer, and in respiratory ailments, arthritis, Rheumatoid Arthritis, Psoriatic Arthritis, Arthritis, Multiple Sclerosis, Fibromyalgia, Psoriasis, Alzheimer's, Asthma, ALS, Tinnitus, Macular Degeneration, Gout, Heart Disease (arteriosclerosis), Autism, to name but a few disease states in which inflammation plays a role and for which the oil disclosed herein is expected to provide therapeutic effects.

The present invention comprises planting seed of Monarda fistulosa or any of the species listed in Table 1 in a field at a rate between about 2.5 lbs per acre and about 5 lbs per acre and more preferably between about 3.5 and 4.5 lbs per acre. Alternatively, plants 14 may be plug planted at a rate between about 24,000 and about 44,000 per acre in either 24 inch or 12 inch rows. The Monarda plants may provide good oil yields for several consecutive years, Harvest may include stems (generally the section above the ground level), along with leaves and seed heads, or may be more limited to harvesting just heads without stems and leaves, or seed heads with leaves.

The equipment used to condition the field, plant the seed (where seeding is employed rather than plug planting), or, alternatively, plant the plugs, mow the plants, cut the plants, and gather the heads and leaves or whole plants may each be of any type readily known and understood by one of skill in the art. Examples of each of these types of equipment are provided herein by machine producer's brand name but they are provided strictly for purposes of example, and not for limitation. Specifically, a Draper head on a forage chopper can be used followed by blowing the foliage into a wagon or other container for moving to the distillery. Seed drills, if used, may be, for example, a Great Plains drill with small seed attachment and 8 inch rows. If broadcasting seed, a Terrogator may be employed.

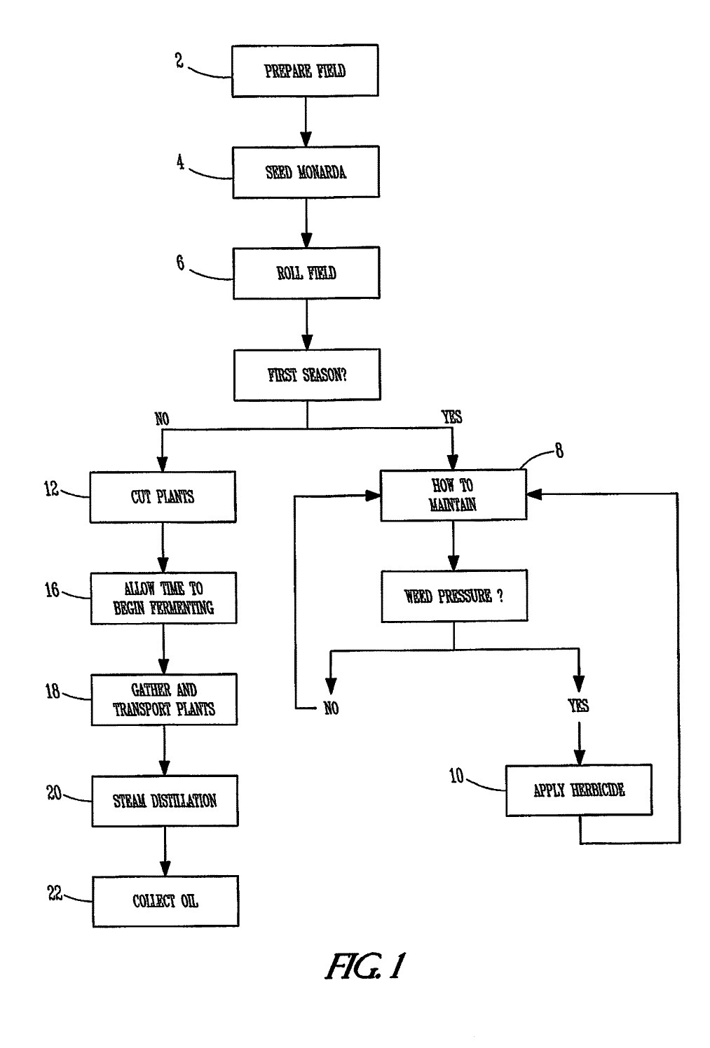

Referring now to FIG. 1 the field 12 is prepared 2 by routine tilling, field cultivating, discing or other means for turning or loosening the upper layer of soil. Next, the field is seeded or plug planted. Seed 10 may be drilled or broadcast. In either case, broadcasting or drilling, seed 10 should be planted at the rates mentioned above. When plug planting, rates suggested are from about 24,000 to about 44,000 in rows spaces between about 12 inches and about 24 inches. Some weeding may be required in the first year. Thereafter, very little weeding is expected to be required as the plants include their own alleopathy as well as shade other smaller plants and aggressively use the nutrients in nearby soil.

Alternatively, and in a preferred method plants may be plug planted at generally even spacing for about 24,000 to about 44,000 plugs per acre. The plugs may be planted in rows or groups or generally equidistant.

During the first season (whether seeded or plug planted), the field 12 may be mowed 8 on a regular basis or, especially where the field is plug planted, will not be mowed the first year. Monarda planted as described herein is unlikely to require any herbicides after the first season, however, Assure II, Basagran and SelectMax are but a few examples of herbicides that may be employed if needed. Monarda fistulosa plants 14 produce their own herbicidal chemicals one of which is carvacrol. These chemicals act to control any weeds that are not simply physically crowded out by the Monarda. The planting rate, the growth rate, the size of the plants, and the Monarda's own chemical defenses result in a crop that typically requires no further herbicide treatments but for, perhaps, an occasional weed patch which may be controlled via hand sprayer or physical removal. This is desirable for the product oil as well as efficiency and cost

The plants may be harvested at flower peak which, in the Midwest United States, is generally early to mid July. The flowers and seed heads contain the essential oil as do the stems and the leaves.

Harvest of Monarda fistulosa fields cultivated in the manner of the present invention may be accomplished in several ways using various equipment. A preferred method is to employ is a MacDon draper or any other cutting machine to cut the plants with flowers attached, transferring the plants, then distilling the oil from the plant parts either within a few hours of harvest or at a later time. Alternatively, the plants and flowers may be left to lay in the field or in a transport container for 2 to up to about 6 or 12 hours-or about 24 hours but doing so may not provide the highest oil yields and may result in contamination of the plant material. Alternatively, the plants (alternatively, the flower heads only, or the flowers and leaves only) may be harvested freshcut and transported directly to the still. In short, the plants do not need to be distilled immediately after harvest which allows for efficient harvest operations when acreage levels and still volumes are not perfectly matched.

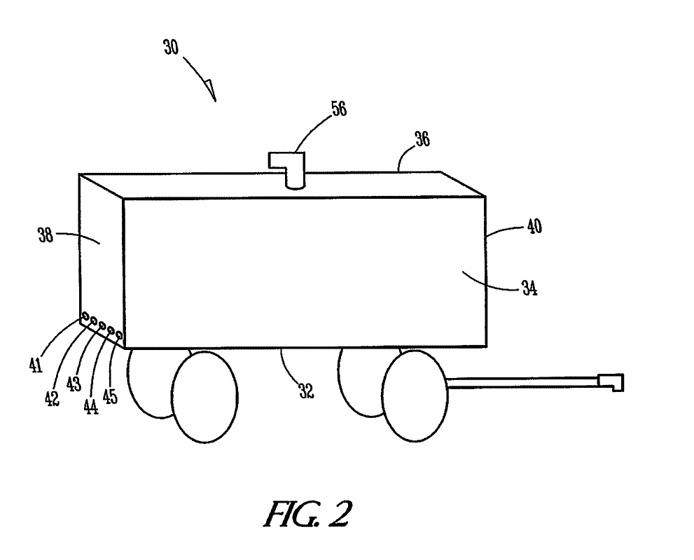

In one embodiment (FIG. 2, 3), means to transport 30 the cut plants comprises a generally watertight wagon having a floor 32, two sides 34, 36, and two ends 38 and 40 with multiple ports 41-45 near the floor 32 in one of the ends 38 or 40. The wagon 30 just described is used in one of several methods for removing oil from Monarda fistulosa, specifically steam distillation.

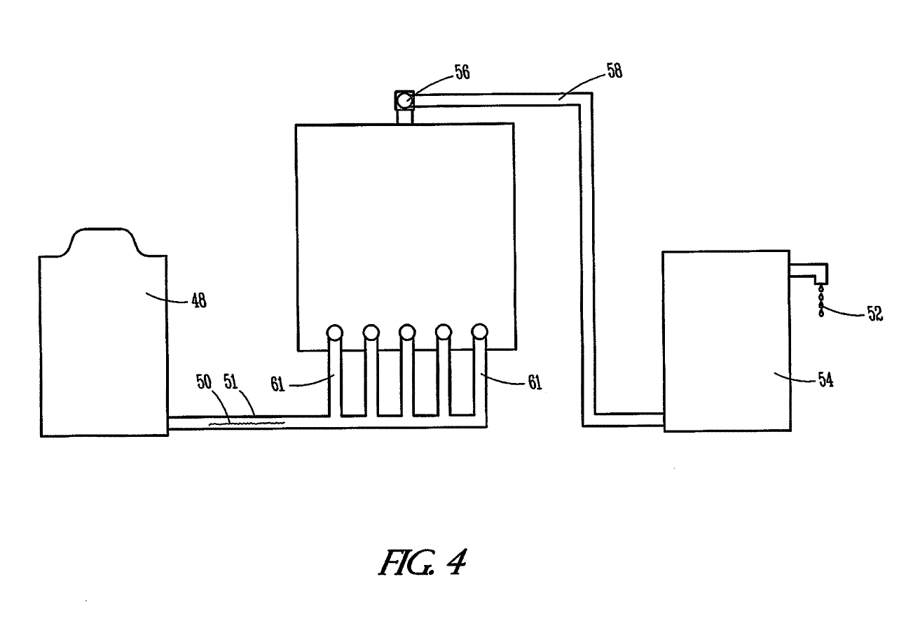

Steam distillation of oil from plants is old in the art and works on the principle that steaming the cut plants (or portions of the cut plants) encourages release of the plants' essential oils via rupture of the plant's oil sacs which are taken up with the steam. During steam distillation 20 a boiler 48 creates steam 50 (see FIG. 4). The steam 50 travels through a conduit 51 to said means to transport 30 (or some other container wherein said plant parts are present) and enters through ports 41-45 near the floor 32. The steam travels upward moving oil 52 that has been released upward with the steam 50. The steam 30, with oil 52, is removed through the port 56 into a second conduit 58. The steam 50 and oil 52 is run through a condenser 54 which causes the oil 52 and water in the steam 50 to condense and allows the oil 52 to be separated and recovered 22. When using the wagon 30, a steam input 61 is connected to each port 41-45. Steam is injected into the wagon and, as it travels upwards, oil 52 from the plants is removed with the steam. The steam and oil then exit the top of the wagon through the port 56 and flow into or through one of many different kinds of condensers 60 where the oil 52 is recovered and the condensed steam (water) may or may not be reheated and reused in the process.

During distillation the essential oils are separated from the distillate in an order characteristic of the oil and related to the boiling point of that oil. (See Table 2 below) Thymoquinone separates just before thymol and carvacrol and thymohydroquinone separates just after carvacrol.

TABLE 2

| compounds | molecular weight | boiling points |
|---|---|---|
| alpha pinene | 136.24 | 155 |
| alpha thujene | 136 | 155 |
| alpha terpinene | 136.237 | 164 |
| beta myrcene | 136.23404 | 167 |
| para cymene | 134.22158 | 179 |
| gamma terpinene | 136.23404 | 182 |
| linalool | 154.252 | 198 |
| terpinene-4-ol | 154.249 | 212 |
| thymoquinone | 164.204 | 231 |
| thymol | 149.66 | 232 |
| carvacrol | 150.221 | 238 |
| caryophyllene | 204.356 | 256 |
| thymohydroquinone | 166.217 | 306 |

The method of cultivation just described increases germination, decreases herbicide use and fuel use, and increases the yield of Monarda fistulosa oil generally, and specifically, effects relative amounts of carvacrol, thymol,and thymoquinone (TQ) and thymohydroquinone (THQ). At least after the first year's growth, and more commonly also after the first year including harvest, the TQ and THQ levels, together, may constitute about 2% to about 35%, and up to about 48% of the oil distilled and either carvacrol or thymol constitute over 20% of the oil distilled, or together constitute over 20% of the oil distilled. The oil content may be more closely predicted by selecting and cloning plants that produce particularly desirable oil profiles and propagating those clones. Some of these clones have been collected and tested to yield 35% to more than 40% thymoquinone. (see table 3 below):

See Table 3 below:

| | YIELD | | |
|---|---|---|---|
| Plant | TQ % | THY % | CARV % |
| 1 | 27.82 | 7.17 | 0.17 |
| 2 | 26.89 | 0.43 | 19.00 |
| 3 | 26.24 | 3.09 | 1.25 |
| 4 | 36.58 | 2.01 | 1.35 |
| 5 | 28.28 | 1.47 | 2.96 |
| 6 | 47.45 | 1.22 | 2.31 |
| 7 | 28.60 | 0.52 | 4.27 |
| 8 | 23.34 | 0.87 | 1.99 |
| 9 | 28.03 | 1.41 | 1.33 |
| 10 | 38.51 | 1.42 | .61 |
| 11 | 27.41 | 6.19 | 1.62 |
| 12 | 39.68 | 4.27 | 1.19 |
| 13 | 31.56 | 4.92 | 0.89 |
| 14 | 24.37 | 29.67 | 0.83 |
| 15 | 20.5 | 29.32 | 1.80 |
| 16 | 29.73 | 12.28 | 1.37 |
| 17 | 26.00 | 1.31 | 7.18 |
| 18 | 22.58 | 10.51 | 5.91 |
| 19 | 24.14 | 0.77 | 1.39 |
| 20 | 37.82 | 2.27 | 6.26 |
| 21 | 22.57 | 1.38 | 0.61 |
| 22 | 29.53 | 0.76 | 1.5 |
| 23 | 23.9 | 0.65 | 0.9 |
| 24 | 23.86 | 8.54 | 2.66 |
| 25 | 28.60 | 0.97 | 1.6 |
| 26 | 29.91 | 2.87 | 3.81 |
| 27 | 29.98 | 9.75 | 1.05 |
| 28 | 15.58 | 0.34 | 0.39 |
| 29 | 32.64 | 6.18 | 0.62 |
| 30 | 32.78 | 5.36 | 0.65 |
| 31 | 31.52 | 0.22 | 1.51 |
| 32 | 24.76 | 10.13 | 2.07 |
| 33 | 34.01 | 0.61 | 3.71 |
| 34 | 30.91 | 8.94 | 2.77 |
| 35 | 24.35 | 18.08 | 1 |
| 36 | 34.12 | 3.5 | 0.87 |
| 37 | 25.83 | 1.57 | 1 |
| 38 | 33.36 | 8.17 | 1.34 |

Certain aspects of the oil profile can be manipulated by the way the Monarda is planted and cultivated as described herein. Combining cloning with the cultivation methods provided herein is well within the scope of the present invention.

The above-mentioned cultivation method results in oil content that is commercially desirable, namely, containing increased amounts of carvacrol, thymol, thymoquinone, and thymohydroquinone as compared to naturally occurring stands of Monarda fistulosa.

Although known recommended planting rates for Monarda fistulosa ranged from about 0.25 to up to 2 lbs per acre (Monarda fistulosa contains 1,120,000 seeds/lb), it was surprisingly found that these rates did not provide the better means for cultivating and harvesting the plants and obtaining the desired essential oils they contained at the level of oil quality and profile desired. Oil quality of the present invention is high at least partially due to the reduction and near elimination of weed pressure and its resulting contamination. The quality may also be partially attributed to low uses of herbicides or pesticides. It is believed also that harvesting methodology at least relative to time between harvest and distillation, and perhaps also relative to plant parts, also attributes to oil profile.

The essential oil operates as an antimicrobial as well as an anti-viral on surfaces. The essential oil of Monarda as described herein is also effective for several health-related purposes, including prevention and therapeutic effect. The oil of Monarda described herein has been shown to have anti-inflammatory affects when applied in any of a wide variety of methods. For example, the oil produced may include from between 2% to about 40% thymoquionone and hydrothymoquinone, combined. The oil can be delivered alone, or with a carrier, or combined with another oil or other components in a composition. It may be delivered in a gel cap, capsule, or a syrup or other edible means for oral dosing. It may be delivered transdermally using a delivery trajectory such as a lotion, cream, transdermal patch; or it may be directly applied to the skin, in either case its cell permeable, lipid soluble, small molecules easily penetrate the skin. It may alternatively be delivered either through mucosal lining or pulmonary avenues as a spray, inhalant, in a vaporized form, or syrup. It may be delivered via medical pump. In short, it may be delivered via several pathways of delivery including transdermal, pulmonary, intravenous, subcutaneous. In general, the essential oil is used to control, prevent, or reduce inflammation in a human or animal, thereby reducing, controlling, or eliminating the symptoms or effects of that inflammation.

Thus, the present invention has been described in an illustrative manner. It is to be understood that the terminology that has been used is intended to be in the nature of words of description rather than of limitation.

Many modifications and variations of the present invention are possible in light of the above teachings. For example, distillation may be accomplished in other ways; seeds may be planted via hand, or via traditional planters, or other seed planting devices not yet known. New effective herbicides may become known. New Monarda hybrids may be developed or clones may be made and it may be determined that other means may be employed to increase oil production even more, specifically the level of carvacrol or of thymoquinone or thymohydroquinone in the Monarda. New transdermal delivery forms, or methods of pulmonary delivery, or oral delivery may be developed; it is expected that some of these new methods may be used to deliver the essential oil of Monarda as described herein for the purposes disclosed. Therefore, within the scope of the appended claims, the present invention may be practiced otherwise than as specifically described.

What I claim is:

1. A therapeutic oil comprising essential oil of monarda, said essential oil of monarda including between about 1% and about 40% thymoquinone and thymohydroquinone combined.

2. A composition comprising the therapeutic oil of claim 1.

3. The composition of claim 2 further comprising at least one of a group consisting of: a second oil, a lotion, a gel, a cream, a paste, an additional therapeutic adjuvant, a food additive, a flavor.

4. The therapeutic oil of claim 1, said oil contained in a gel cap.

5. The therapeutic oil of claim 1, said oil delivered by at least one delivery trajectory selected from the group consisting of: a lotion, a cream, a gel, a transdermal patch, a spray, an injectable adjuvant, an intravenous solution.

6. A method of delivering the therapeutic oil of claim 1 to a mammal, said method selected from a group consisting of: transdermal application, pulmonary delivery, oral ingestion, intravenous drip, subcutaneous injection, subcutaneous implant, and medical pump.

7. The composition of claim 2, said composition comprising a second oil.

8. The method of claim 6 wherein pulmonary delivery comprises delivery of the therapeutic oil by one of a group consisting of: an inhaler, an inhalant, a diffuser.

9. The therapeutic oil of claim 1, said oil obtained by steam distillation of a plurality of leaves and seed heads harvested from Monarda fistulosa plants grown from plugs planted between about 24,000 and 60,000 plants per acre.

10. A method of managing inflammation in a mammal, said method comprising using essential oil of monarda comprising between about 1% and 40% thymoquinone and thymohydroquinone combined.

11. The method of claim 10 comprising administering an amount of said essential oil to a human by one from the following methods: transdermal, subcutaneous, intravenous, inhalation, pulmonary delivery, oral ingestion.

12. The method of claim 11 wherein administration is transdermal comprising applying the essential oil to a portion of the sole of a human foot.

13. The method of claim 12 wherein the essential oil is combined with at least one of the following prior to transdermal application: a second oil, lotion, cream, gel.

14. The method of claim 11 wherein administration is by oral ingestion comprising ingesting a gel cap containing a composition of the essential oil of Monarda.

15. The method of claim 11 wherein administration is by inhalation comprising diffusing said essential oil prior to inhalation.

16. The method of claim 10 wherein the inflammation is caused by arthritis.

17. The method of claim 10 wherein the inflammation is caused by cancer.

18. The method of claim 10 wherein the inflammation is caused by a viral infection.

19. The method of claim 10 wherein the inflammation is caused by a microbial infection.

20. The method of claim 10 wherein a symptom of the inflammation is respiratory.

21. The method of claim 10 wherein a symptom of the inflammation is tumor growth.

22. The method of claim 10 wherein a symptom of the inflammation is related to diet, lack of exercise and genetics.

23. The method of claim 10 wherein the inflammation is caused by cancer.

24. The method of claim 23 wherein the cancer is leukemia.

25. The composition of claim 2, said composition contained in a gel cap.

26. The composition of claim 2, said composition delivered by at least one delivery trajectory selected from the group consisting of: a lotion, a cream, a gel, a transdermal patch, a spray, an injectable adjuvant, an intravenous solution.

27. A method of delivering the composition of claim 2 to a human said method selected from a group consisting of: transdermal application, pulmonary delivery, oral ingestion, intravenous drip, subcutaneous injection, subcutaneous implant, and medical pump.

28. A method of managing inflammation in a human comprising using the composition of claim 2.

29. The method of claim 6 wherein said mammal includes humans and companion animals.
